(19) United States
(12) Patent Application Publication
(10) Pub. No.: US 2006/0074492 A1
(43) Pub. Date: Apr. 6, 2006

(54) Endoprosthesis for a metatarsophalangeal joint

(76) Inventor: Theo Frey, Mettmenstetten (CH)

Correspondence Address: KNOBBE MARTENS OLSON & BEAR LLP, 2040 MAIN STREET, FOURTEENTH FLOOR, IRVINE, CA, 92614, US

(21) Appl. No.: 11/222,678

(22) Filed: Sep. 9, 2005

(30) Foreign Application Priority Data
Sep. 9, 2004 (DE) DE102004043700.9

(51) Int. Cl. A61F 2/42

(52) U.S. Cl. 623/21.15; 623/21.19

(57) ABSTRACT

An endoprosthesis for a metatarsophalangeal (MTP) joint, especially a basal joint of a large toe, a toe joint or a finger joint, has a prosthesis-half defining a concave or convex prosthetic sliding surface, and means arranged eccentrically with respect to the prosthetic sliding surface of the prosthesis-half for anchoring the latter in the bone.

### CROSS-REFERENCE TO RELATED APPLICATIONS

This application claims priority to German Patent Application No. DE 102004043700.9, filed Sep. 9, 2004, the entire contents of which are hereby incorporated by reference and should be considered a part of this specification.

### BACKGROUND OF THE INVENTION

1. Field of the Invention

The invention relates to an endoprosthesis for a joint, and in particular to an endoprosthesis for a metatarsophalangeal (MTP) joint, especially a basal joint of a large toe, a toe joint or a finger joint, having a prosthesis-half defining a concave or convex prosthetic sliding surface.

2. Description of the Related Art

A prosthesis for an MTP joint is described, for example, in EP 0 827 386 B1. In addition thereto, it should be stated that the joint replacement for a basal joint of a toe, especially a large toe, nowadays consists of replacing the joint surface either distally (proximal phalanx) by a prosthesis-half or hemiprosthesis or distally and proximally (proximal phalanx and metatarsal) with a total prosthesis. Both monoblock and modular endoprostheses are used in the process. The artificial joint replacement is usually made of metal, cobalt chromium alloys or titanium alloys or pure titanium, although ceramic material or polyethylene are also used. Modular endoprostheses for the large toe are anchored in the bone, mainly in a cementless manner, such as via a so-called “press fit” connection or with the use of screws.

The proximal component of a total prosthesis has an axially centered or cranked joint surface replacement in the form of a spherical segment. The distal component has a disc centrally arranged with respect to the axis of the anchoring pin or stem of the distal component. The disc has a depression or socket which acts as a sliding bearing for the proximal component.

However, it is intraoperatively very difficult to center the implant in the bone. For example, the joint insert can form an overhang, which results in irritation of soft tissue or of the joint capsule. The overhang can also cause a reduction in the range of movement because the raising of the implant can result in relatively high soft tissue tension. In extreme cases, dislocation can occur.

Accordingly, there is a need to provide an endoprosthesis in which the mentioned overhang, especially in the dorsal or plantar region of the end of the metatarsal or phalangeal bone, can be avoided.

### SUMMARY OF THE INVENTION

One aspect of the invention is the realization that it should be possible, by means of a centered alignment of the metatarsal to the proximal phalanx or vice-versa, to reduce the risk of dislocation. Another aspect of the invention is the realization that the endoprosthesis should be constructed so that it is possible to dispense with complicated target guidance instruments. In particular, it should be possible for the parts of the implant to be implanted in a non-centered position and to subsequently adjust said parts to fit them to the particular patient.

In one embodiment, an endoprosthesis for a joint selected from the group consisting of a metatarsophalangeal (MTP) joint, a basal joint of a large toe, a toe joint, and a finger joint is provided. The endoprosthesis comprises at least one prosthesis-half defining a prosthetic sliding surface selected from the group consisting of a concave surface, and a convex surface. The prosthesis-half includes eccentric means arranged eccentrically with respect to the prosthetic sliding surface of said prosthesis-half and operative to anchor the latter in the bone.

In another embodiment, an endoprosthesis for a joint selected from the group consisting of a metatarsophalangeal (MTP) joint, a basal joint of a large toe, a toe joint, and a finger joint is provided. The endoprosthesis comprises a prosthesis-half defining a prosthetic sliding surface about a first axis and an anchor connected to the prosthesis-half and extending about a second axis, the anchor configured to anchor the prosthesis-half in a bone of a joint. The first and second axes are offset by a predetermined amount.

In another embodiment, a prosthesis for a joint selected from the group consisting of a metatarsophalangeal (MTP) joint, a basal joint of a large toe, a toe joint, and a finger joint is provided. The prosthesis comprises a first prosthesis-half defining a first prosthetic sliding surface about a first axis and a first anchor extending about a second axis, the anchor configured to anchor the first prosthesis-half in a bone of a joint. The prosthesis also comprises a second prosthesis-half defining a second prosthetic sliding surface about a third axis and a second anchor extending about a fourth axis, the second anchor configured to anchor the second prosthesis-half in a second bone of the joint. The first and second prosthetic sliding surfaces are configured to slidingly engage each other, wherein at least one of the first and third axes is offset relative to the corresponding second or fourth axis by a predetermined amount.

In another embodiment, a method for implanting an endoprosthesis in a bone of a joint selected from the group consisting of a metatarsophalangeal (MTP) joint, a basal joint of a large toe, a toe joint, and a finger joint is provided. The method comprises providing a prosthesis-half having a prosthetic sliding surface and an anchor, implanting the prosthesis-half into a bone of a joint, intraoperatively finding an optimum orientation of the prosthetic sliding surface, and finalizing the anchoring of the prosthesis-half in the bone.

In another embodiment, a method for implanting an endoprosthesis in a bone of a joint selected from the group consisting of a metatarsophalangeal (MTP) joint, a basal joint of a large toe, a toe joint, and a finger joint is provided. The method comprises providing a prosthesis-half having a prosthetic sliding surface about a first axis and an anchor about a second axis, the second axis being offset relative to the first axis. The method also comprises implanting the prosthesis-half into a bone of a joint, rotating the prosthesis-half so as to adjust the orientation of the prosthetic sliding surface, and fixing the prosthesis-half to the bone.

### BRIEF DESCRIPTION OF THE DRWINGS

Preferred embodiments of the endoprosthesis according to the invention are described in greater detail hereinbelow with reference to the accompanying drawings, in which:

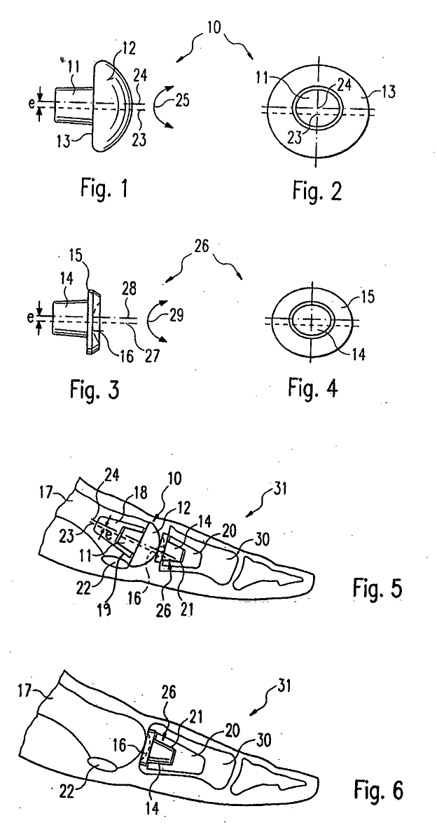

FIG. 1 is a side view of one embodiment of a proximal prosthesis-half.

FIG. 2 shows a bottom view of the proximal prosthesis-half according to FIG. 1, that is to say, from the proximal direction.

FIG. 3 is a side view of one embodiment of a distal prosthesis-half.

FIG. 4 shows a bottom view of the distal prosthesis-half according to FIG. 3, that is to say, from the distal direction.

FIG. 5 is a side view of one embodiment of a total prosthesis having an eccentrically positioned proximal prosthesis-half.

FIG. 6 is a side view of one embodiment of a hemiprosthesis having an eccentrically positioned distal prosthesis-half.

### DETAILED DESCRIPTION OF THE PREFERRED EMBODIMENTS

FIGS. 1 and 2 show one embodiment of a proximal joint insert or proximal prosthesis-half 10. In the illustrated embodiment, the proximal prosthesis-half 10 comprises a convex prosthetic sliding surface 12 with an anchoring pin 11 of conical shape, which is arranged eccentrically with respect to a central axis 23 of the sliding surface 12. However, one of ordinary skill in the art will recognize that the prosthetic sliding surface 12 can have other shapes, such as concave. Likewise, the anchoring pin 11 can have other suitable shapes, such as cylindrical. The proximal prosthesis-half 10 also defines a rear side or surface 13, which is located opposite the prosthetic sliding surface 12. Reference numeral 24 denotes a central axis of the anchoring pin 11. In the illustrated embodiment, the central axes 23, 24 are offset from one another by a predetermined eccentricity “e”. In one preferred embodiment, the eccentricity “e” is between about 0.5 mm and about 5.0 mm. In another preferred embodiment, the eccentricity “e” is between about 0.8 mm and about 3.0 mm. Consequently, the prosthetic sliding surface 12 can be rotated back and forth, e.g. in the direction of the double-headed arrow 25 shown in FIG. 1, about the central axis 24 of the anchoring pin 11, until the suitable or optimum rotational position of the prosthetic sliding surface 12 relative to a corresponding prosthesis-half or a bone, as described below, has been found intraoperatively. In one preferred embodiment, once said optimum rotational position has been found, the definitive anchoring or coupling of the prosthesis-half or joint insert 10 is carried out.

FIGS. 3 and 4 show a distal prosthesis-half 26 of complementary form to the proximal prosthesis-half 10 shown in FIGS. 1 and 2. The prosthesis-halves 10, 26 can be of monoblock or, alternatively, modular construction. In the illustrated embodiment, the distal prosthesis-half 26 comprises a concave prosthetic sliding surface 16 having a central axis 27, and a conical anchoring pin 14 having a central axis 28. However, the prosthetic sliding surface 16 can have other suitable shapes, such as convex. Likewise, the anchoring pin 14 can have other shapes, such as cylindrical. The distal prosthesis-half 26 also defines a rear side or surface 15, which is located opposite the prosthetic sliding surface 16. The two central axes 27, 28 are offset from one another in like manner to the central axes 23, 24 of the proximal prosthesis-half. This offset defines a predetermined eccentricity “e”.

In one preferred embodiment, prior to definitive locking of the distal joint insert or distal prosthesis-half 26, the prosthesis-half 26 can be rotated back and forth, e.g., in the direction of the double-headed arrow 29 shown in FIG. 3, about the central axis 28 of the pin until an optimum rotational position of the associated prosthetic sliding surface 16 has been reached. Definitive locking is then carried out in the bone, or in a connection element associated with the prosthesis-half 26 and which is anchored in the bone. The connection element can be anchored in the bone by cementless means or by cementing, as further discussed below.

FIG. 5 shows one embodiment of a total prosthesis for an MTP joint in an X-ray-like side view. In the illustrated embodiment, the prosthesis is disposed in a phalanx bone 30 and a metatarsal bone 17, proximal a sesamoid bone 22, of a large toe 31. Fixed to the distal end of the metatarsal 17 is the spherical cap-like proximal prosthesis-half 10 with the corresponding prosthetic sliding surface 12, which is in corresponding arrangement with the complementary concave sliding surface 16 of the distal prosthesis-half 26, which is implanted at a proximal end of the phalanx 30.

In the illustrated embodiment, the proximal prosthesis-half 10 is anchored using a connection element 18 implanted at the distal end of the metatarsal 17. The prosthesis-half 10 is preferably anchored in the connection element 18 via a self-limiting plug connection between the conical anchoring pin 11 and a complementarily conical pin receiving bore 19. The connection element 18 is anchored within the metatarsal bone 17 by cementless means. However, the connection element 18 can alternatively be anchored within the metatarsal bone 17 by cementing, in any suitable manner known in the art. In addition thereto, reference may also be made to EP 0 827 386 B1, which has already been mentioned at the beginning, and which is hereby incorporated by reference in its entirety and should be considered a part of this specification.

The desirable aspect is the interplay between the pin receiving bore 19 and the anchoring pin 11, which in the illustrated embodiment is eccentrically arranged relative to the prosthetic sliding surface 12. In one embodiment, the pin receiving bore 19 is co-axial with the connection element 18, so that the receiving bore 19 and the connection element 18 extend about the same axis (i.e., the receiving bore 19 is centrally located in the connection element 18). In another embodiment, the pin receiving bore 19 can be eccentrically prepared relative to the connection element 18, so that the receiving bore 19 extends about an axis that is offset by a predetermined eccentricity “e2” relative to an axis about which the connection element 18 extends. In one embodiment, the eccentricity “e2” is equal to the eccentricity “e” between the prosthetic sliding surface 12 and the anchoring pin 11. However, the eccentricity “e2” can also differ from the eccentricity “e” in the proximal prosthesis-half 10. In one preferred embodiment, the eccentricity “e2” is between about 0.3 mm and about 1.5 mm.

As shown in FIG. 5, the complementary distal prosthesis-half 26 can also be anchored in the phalanx bone 30 in the same manner. In one preferred embodiment, a separate connection element 20 is implanted in the phalanx bone 30. Preferably, the distal joint insert or prosthesis-half 26 can be coupled to the connection element 20, via the interaction between the anchoring pin 14 and a pin receiving bore 21 of the connection element 20. In the illustrated embodiment, the pin receiving bore 21 has a conical shape corresponding to the conical shape of the anchoring pin 14. In one embodiment, the prosthesis-half 26 has an eccentric anchoring pin 14 relative to the sliding surface 16, as discussed above. In another embodiment, the distal prosthesis-half 26 has an anchoring pin 14 that is co-axial (i.e., not eccentric) relative to the prosthetic sliding surface 16. In one embodiment, the pin receiving bore 21 can be prepared so as to have an eccentricity “e2” relative to the connection element 20, as discussed above.

In one embodiment, as previously discussed, the prosthesis-half 26 can be anchored to the connection element 20 via a self limiting plug connection between the anchoring pin 14 and the pin receiving bore 21. However, the prosthesis-half 10, 26 can be anchored in the connection element 18, 20 via other mechanisms, such as a screw. For example, the prosthesis-half 10, 26, or a fixation part thereof, can have a rectilinearly or arcuately extending slot-like through-hole for receiving a fixation screw, the through-hole preferably being located away from the orthopaedically active prosthetic sliding surface 12, 16. In this embodiment, the connection element 18, 20 could have a threaded bore for coupling engagement with said screw. In another embodiment, the connection element 18, 20 can have a plurality of threaded bores spaced apart from one another for accommodation of a fixation screw associated with the prosthesis-half 10, 26, the threaded bores being arranged so that the prosthesis-half 10, 26 can be fixed to the connection element 18, 20 with the prosthetic sliding surface 12, 16 of the former either centrally or, if so required, eccentrically arranged with respect to the central axis of the latter. In still another embodiment, the prosthesis-half 10, 26 can have a somewhat larger through-hole or a somewhat wider slot, compared to the diameter of the fixation screw, so that prior to definitive locking by means of the fixation screw the prosthesis-half 10, 26 can be moved intraoperatively in all directions, more particularly into a centered or eccentric position, as desired.

In one embodiment, the pin receiving bores 19, 21 can be formed eccentrically within the connection elements 18, 20, as discussed above, in which case the eccentricity is preferably the same as that between the prosthetic sliding surfaces 12, 16 on the one hand and the anchoring pins 11, 14 on the other hand. However, the anchoring pin 14 of the distal joint insert 26 can be arranged co-axially (i.e. not arranged eccentrically) with respect to the associated prosthetic sliding surface 16.

In one embodiment, the central axis 23, 27 of the prosthesis sliding surface 12, 16 extends generally parallel to the corresponding central axis 24, 28 of the anchoring pin 11, 14. In another embodiment, the central axis 23, 27 of the prosthesis sliding surface 12, 16 extends at a predetermined angle to the corresponding central axis 24, 28 of the anchoring pin 11, 14, such as an acute angle. Accordingly, it is possible to correct a position of the large toe without requiring onerous intracapital wedge osteotomy or basal wedge osteotomy.

FIG. 6 shows one embodiment of a hemiprosthesis having a distal joint insert or prosthesis-half 26 implanted at the proximal end of the phalanx bone 30, such as that shown in FIG. 5. The prosthesis-half 26 has a concave prosthetic sliding surface 16. In the illustrated embodiment, the prosthesis-half 26 corresponds to the embodiment shown in FIGS. 3 and 4 and has an anchoring pin 14 arranged eccentrically with respect to the prosthetic sliding surface 16. The anchoring pin 14 is preferably in corresponding arrangement with a complementary pin receiving bore 21 of a previously implanted connection element 20. As illustrated in FIG. 6, the prosthesis-half 26 need not engage a corresponding prosthesis-half, but rather, can engage an adjacent bone itself.

One of ordinary skill in the art will recognize that the described arrangement can also be implemented at other toe joints or finger joints. Use of the described invention is not to be limited in that respect. It should also be mentioned that, in the case of the implant for the phalanx or basal phalanx 30, different plateau heights are possible, as a result of which a balanced capsule tension can be restored or obtained.

Advantageously, the embodiments of the prosthesis-halves 10, 26 discussed above have anchors 11, 14 allowing the prosthesis-halves 10, 26 to be implanted in bone. The anchors 11, 14 are advantageously arranged so that the concave or convex prosthetic sliding surfaces 12, 16 can be brought into either a centered position or, if so required, an eccentric position. Accordingly, it is possible for the associated prosthetic sliding surfaces 12, 16 to be adjusted subsequent to implantation and adapted to the particular patient, even when a prosthesis-half 10, 26 has been implanted in a non-centered position. Additionally, the prosthesis-half 10, 26 can advantageously be adjusted subsequent to implantation without need for special instruments. The eccentricity provided in accordance with the embodiments discussed above allows subsequent adjustment of a prosthesis-half 10, 26 by means of simple rotation until the desired position has been reached and prior to carrying out definitive coupling of the prosthesis-half 10, 26 with the bone or to a connection element 18, 20 previously anchored in the bone by cementless means or by cementing. The adjustment of the prosthesis-half 10, 26 or of the concave or convex prosthetic sliding surface 12, 16 thereof can thus be advantageously carried out intraoperatively by the surgeon.

All features disclosed in the application documents are claimed as being important to the invention insofar as they are novel on their own or in combination compared with the prior art.

While certain embodiments of the inventions have been described, these embodiments have been presented by way of example only, and are not intended to limit the scope of the inventions. Indeed, the novel methods and systems described herein may be embodied in a variety of other forms. For example, the foregoing may be applied to the implantation of endoprostheses of bones other than the phalanx or metatarsal bones, or joints other than a big toe. Furthermore, various omissions, substitutions and changes in the form of the methods and systems described herein may be made without departing from the spirit of the inventions. The accompanying claims and their equivalents are intended to cover such forms or modifications as would fall within the scope and spirit of the inventions.

### REFERENCE SYMBOLS

10 proximal joint insert or proximal prosthesis-half
11 anchoring pin
12 convex prosthetic sliding surface
13 rear surface
14 anchoring pin
15 rear surface
16 concave prosthetic sliding surface
17 metatarsal
18 connection element
19 pin receiving means
20 connection element
21 pin receiving means
22 sesamoid bone
23 central axis
24 central axis
25 double-headed arrow
26 distal joint insert or distal prosthesis-half
27 central axis
28 central axis
29 double-headed arrow
30 (basal) phalanx
31 large toe joint
e eccentricity

What is claimed is:

1. An endoprosthesis for a joint selected from the group consisting of a metatarsophalangeal (MTP) joint, a basal joint of a large toe, a toe joint, and a finger joint, said endoprosthesis comprising at least one prosthesis-half defining a prosthetic sliding surface selected from the group consisting of a concave surface, and a convex surface, said prosthesis-half including eccentric means arranged eccentrically with respect to the prosthetic sliding surface of said prosthesis-half and operative to anchor the latter in the bone.

2. An endoprosthesis according to claim 1, wherein each prosthesis-half has an anchoring pin operative to anchor said prosthesis-half, said anchoring pin having a central axis offset relative to the central axis of said respective concave or convex prosthetic sliding surface.

3. An endoprosthesis according to claim 1, said endoprosthesis being of modular construction and further including, in addition to said prosthesis-half having said prosthetic sliding surface, a connection element operative to connect said prosthesis-half, said connection element being adapted to be anchored in the bone by anchoring means selected from the group consisting of cementless means and cementing means.

4. An endoprosthesis according to claim 3, wherein said prosthesis-half is releasably fixed to said connection element by means selected from the group consisting of screws, and a self-limiting plug connection.

5. An endoprosthesis according to claim 4, wherein said self-limiting plug connection is formed by a conical anchoring pin and a complementarily conical pin receiving means, the central axis of means selected from the group consisting of said pin, and said pin and said pin receiving means being offset relative to the central axis of said respective concave or convex prosthetic sliding surface.

6. An endoprosthesis according claim 1, wherein the central axes of said prosthetic sliding surface and prosthesis anchor extend at an orientation selected from the group consisting of parallel, and at a predetermined angle to one another.

7. An endoprosthesis according to claim 3, wherein said prosthesis-half is fixed to the connection element by a screw fixing means and said prosthesis-half or a fixation part thereof has a through-hole selected from the group consisting of a rectilinearly extending slot-like through-hole, and an arcuately extending slot-like through-hole to accommodate a fixation screw, said through-hole preferably being located away from said orthopaedically active prosthetic sliding surface.

8. An endoprosthesis according to claim 3, wherein said prosthesis-half is fixed to the connection element by a screw fixing means, and said connection element has a plurality of threaded bores spaced apart from one another to accommodate a fixation screw associated with said prosthesis-half so that said prosthesis-half can be fixed to the connection element with said prosthetic sliding surface of the former either centrally or, if so required, eccentrically arranged with respect to the central axis of the latter.

9. An endoprosthesis according to claim 7, wherein the through-hole for said fixation screw of said prosthesis-half is dimensioned larger than the diameter of said fixation screw or of the shank thereof so that prior to definitive locking by means of said fixation screw said prosthesis-half can be moved towards all sides into a centered or eccentric position.

10. An endoprosthesis for a joint selected from the group consisting of a metatarsophalangeal (MTP) joint, a basal joint of a large toe, a toe joint, and a finger joint, comprising:
a prosthesis-half defining a prosthetic sliding surface about a first axis; and
an anchor connected to the prosthesis-half and extending about a second axis, the anchor configured to anchor the prosthesis-half in a bone of a joint,
wherein the first and second axes are offset by a predetermined amount.

11. The endoprosthesis of claim 10, wherein the first and second axes extend generally parallel to each other.

12. The endoprosthesis of claim 10, wherein the first and second axes extend at an acute angle relative to each other.

13. The endoprosthesis of claim 10, further comprising a connection element configured to be implanted in the bone, the connection element having a bore configured to couplingly receive the anchor therein.

14. The endoprosthesis of claim 10, wherein the predetermined amount is between about 0.5 mm and about 5.0 mm.

15. The endoprosthesis of claim 10, wherein the predetermined amount is between about 0.8 mm and about 3.0 mm

16. A prosthesis for a joint selected from the group consisting of a metatarsophalangeal (MTP) joint, a basal joint of a large toe, a toe joint, and a finger joint, said prosthesis comprising:
a first prosthesis-half defining a first prosthetic sliding surface about a first axis and a first anchor extending about a second axis, the anchor configured to anchor the first prosthesis-half in a bone of a joint; and
a second prosthesis-half defining a second prosthetic sliding surface about a third axis and a second anchor extending about a fourth axis, the second anchor configured to anchor the second prosthesis-half in a second bone of the joint,
wherein first and second prosthetic sliding surfaces are configured to slidingly engage each other, and wherein at least one of the first and third axes is offset relative to the corresponding second or fourth axis by a predetermined amount.

17. The prosthesis of claim 16, wherein the first prosthetic sliding surface is convex and the second prosthetic sliding surface is concave.

18. The prosthesis of claim 16, further comprising:
a first connection element configured to be implanted in the bone, the first connection element having a bore configured to couplingly receive the first anchor therein; and
a second connection element configured to be implanted in the second bone, the second connection element having a bore configured to couplingly receive the second anchor therein.

19. The prosthesis of claim 16, wherein the predetermined amount is between about 0.3 mm and about 5.0 mm.

20. The prosthesis of claim 16, wherein the predetermined amount is between about 0.8 mm and about 3.0 mm.

21. A method for implanting an endoprosthesis in a bone of a joint selected from the group consisting of a metatarsophalangeal (MTP) joint, a basal joint of a large toe, a toe joint, and a finger joint, comprising:
providing a prosthesis-half having a prosthetic sliding surface and an anchor;
implanting the prosthesis-half into a bone of a joint;
intraoperatively finding an optimum orientation of the prosthetic sliding surface; and
finalizing the anchoring of the prosthesis-half in the bone.

22. The method of claim 21, wherein implanting the prosthesis-half includes positioning a connection element into the bone, said connection element couplingly receiving the prosthesis-half therein.

23. The method of claim 22, wherein couplingly receiving includes coupling via a self-limiting connection.

24. The method of claim 21, wherein intraoperatively finding includes rotating the prosthesis-half so as to adjust the orientation of the prosthetic sliding surface between a centered position and an eccentric position relative to a counterpart sliding surface.

25. A method for implanting an endoprosthesis in a bone of a joint selected from the group consisting of a metatarsophalangeal (MTP) joint, a basal joint of a large toe, a toe joint, and a finger joint, comprising:
providing a prosthesis-half having a prosthetic sliding surface about a first axis and an anchor about a second axis, the second axis being offset relative to the first axis;
implanting the prosthesis-half into a bone of the joint;
rotating the prosthesis-half so as to adjust the orientation of the prosthetic sliding surface; and
fixing the prosthesis-half to the bone.

26. The method of claim 25, wherein implanting the prosthesis-half includes positioning a connection element into the bone, said connection element couplingly receiving the prosthesis-half therein.

27. The method of claim 25, wherein said prosthesis-half is a proximal prosthesis half implanted into a first bone, and further comprising:
providing a distal prosthesis-half having a prosthetic sliding surface about a third axis and an anchor about a fourth axis, the fourth axis being offset relative to the third axis;
implanting the distal prosthesis-half into a second bone of the joint;
rotating the distal prosthesis-half so as to adjust the orientation of the prosthetic sliding surface of the distal prosthesis-half; and
fixing the distal prosthesis-half to the bone;
wherein the sliding surface of the proximal prosthesis-half is provided in complementary engagement with the sliding surface of the distal prosthesis-half

28. The method of claim 27, wherein implanting the proximal and distal prosthesis-halves includes positioning a first and second connection elements into the first and second bones, respectively, said connection elements couplingly receiving the proximal and distal prosthesis-halves, respectively, therein.

29. The method of claim 27, wherein the proximal prosthesis-half is implanted into the metatarsal and the distal prosthesis-half is implanted into the phalanx.

30. The method of claim 27, wherein the sliding surface of the proximal-prosthesis-half is convex and the sliding surface of the distal prosthesis-half is concave.
